(19) United States
(12) Patent Application Publication
(10) Pub. No.: US 2011/0151446 A1
(43) Pub. Date: Jun. 23, 2011

(54) CELL TREATMENT SOLUTION AND METHOD OF PREPARING STAINED CELL SUSPENSION FOR A MEASUREMENT OF NUCLEAR DNA BY FLOW CYTOMETRY

(75) Inventors: Takahiro SHIOYAMA, Tokyo (JP); Sunao TAKEDA, Tokyo (JP); Akane SUZUKI, Tokyo (JP); Naoki KOBAYASHI, Tokyo (JP); Yuko NAGAI, Tokyo (JP)

(73) Assignee: Nihon Kohden Corporation, Tokyo (JP)

(21) Appl. No.: 12/702,262

(22) Filed: Feb. 8, 2010

(30) Foreign Application Priority Data
Feb. 9, 2009 (JP) 2009-27339
Oct. 23, 2009 (JP) 2009-244702

(51) Int. Cl. C12Q 1/68

(52) U.S. Cl. 435/6.1

(57) ABSTRACT

A cell treatment solution and a method that is used for preparing a stained cell suspension that is provided to a measurement of nuclear DNA by flow cytometry. The cell treatment solution may include a surfactant, RNase, and a fluorescent dye. The surfactant may include, for example, a non-ionic surfactant, a zwitterionic surfactant, an anionic surfactant, and/or a cationic surfactant. In one method of the invention, stained cell suspension that is provided to a measurement of nuclear DNA by flow cytometry is prepared. The method may include adding a tissue sample to a cell treatment solution including a surfactant, RNase, and fluorescent dye, disaggregating the tissue sample, and filtering the disaggregated tissue sample. Another method of the invention includes disaggregating a tissue sample, preparing cell suspension by filtering the disaggregated tissue sample, and adding a cell treatment solution including a surfactant, RNase, and fluorescent dye.

This application claims priority to Japanese Patent Applications No. 2009-27339 filed on Feb. 9, 2009 and No. 2009-244702 filed on Oct. 23, 2009 in the Japan Patent Office, the disclosure of which is incorporated herein in its entirety by reference.

### BACKGROUND OF THE INVENTION

1. Field of the Invention

The present invention relates to a cell treatment solution and method of use thereof, and more particularly to a cell treatment solution and a method that is used for preparing a stained cell suspension that is provided to a measurement of nuclear DNA by flow cytometry.

2. Description of the Related Art

In the related art, when the amount of nuclear DNA is measured by flow cytometry, the following steps need to be carried out. First, a tissue sample is mechanically disintegrated, and a cell suspension is filtered by a mesh with a prescribed mesh diameter. Secondly, nuclei are isolated with a surfactant, and RNA is then removed with an RNA removing solution. Finally, nuclear DNA is stained with a fluorescent dye.

In this measurement method, the above-mentioned nuclei isolation with a surfactant, RNA removal with an RNA removing solution, and fluorescent staining with a fluorescent dye are sequentially carried out such as described in Japanese Tokuhyo Patent Application No. Hei 9[1997]-509496

However, sequentially carrying out three processes of nuclei isolation with a surfactant, RNA removal with an RNA removing solution, and fluorescent staining with a fluorescent dye can be time consuming, sometimes requiring approximately 30 minutes, making this process burdensome and time consuming to a measurer.

The present invention considers the above-mentioned current situation in the measurement of nuclear DNA by flow cytometry, and provides a cell treatment solution and a method simplifying the steps and shortening the time for preparing a stained cell suspension that is used for the measurement of the amount of nuclear DNA by flow cytometry, thereby reducing the time of this process.

### SUMMARY OF THE INVENTION

In order to solve the problem mentioned above, in exemplary embodiments of the present invention, instead of carrying out the three steps with the three different solutions in order to prepare a stained cell suspension from a cell suspension as in the related art, a method of the present invention may include only a single step using a cell treatment solution comprising surfactant, RNase and fluorescent dye.

In an aspect of the invention, the surfactant may include, for example, a non-ionic surfactant and a zwitterionic surfactant which may be most effective, and an anionic surfactant and a cationic surfactant which will resolve a cell membrane, but will not damage nucleus DNA that could be used. In exemplary embodiments, the surfactant may include one of Triton X-100, Tween 20 (polyoxyethylene sorbitan monolaurate), and NP-40 (polyoxyethylene (9) octylphenyl ether) or a combination thereof. The fluorescent dye may include a propidium iodide in phosphate buffer. An exemplary embodiment composition of the cell treatment solution may be 0.03% Triton X-100, 0.03% RNase and 60 ug/ml propidium iodide in phosphate buffer. Further, the solution may be freeze-dried and stored for future use.

An exemplary method of preparing a stained cell suspension according to the present invention that is provided to a measurement of nuclear DNA by flow cytometry comprises a cell isolation process through a mechanical disaggregating that puts tissues into a buffer solution and brakes up the tissues at a prescribed number of rotation for a prescribed time; a filtration process that filters out larger pieces of the tissue which were generated in the cell isolation process through the mechanical disintegrating, by a mesh with a prescribed mesh diameter; and a staining process that simultaneously isolates nuclei, removes RNA and stains nuclear DNA by adding and mixing with the cell treatment solution. The stained cell suspension is provided to a measurement of nuclear DNA by flow cytometry.

In embodiments using the freeze-dried cell treatment solution, the method above may further comprise the step of a cell treatment solution preparation process by adding a buffer solution to the freeze-dried cell treatment solution.

In yet another aspect of the invention, an exemplary method of preparing a stained cell suspension that is provided to a measurement of nuclear DNA by flow cytometry comprises a staining process that simultaneously isolates nuclei, removes RNA, and stains nuclear DNA by adding a tissue sample to the cell treatment solution, a cell isolation process by mechanically disaggregating the tissue, and a filtration process that filters off larger pieces.

In embodiments using the freeze-dried cell treatment solution, the method above may further comprise the step of a cell treatment solution preparation process by adding a buffer solution to the freeze-dried cell treatment solution.

Accordingly, by using either the cell treatment solution or the freeze-dried cell treatment solution and the methods of the present invention, the process of preparing a stained cell suspension for a measurement of nuclear DNA by flow cytometry becomes much simpler and requires much less time, reducing potential errors and mishandlings during the measurement.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 is a flow chart showing a method of protocol of measuring the amount of nuclear DNA by using a cell treatment solution according to an exemplary embodiment of the invention;

FIG. 2 depicts the results obtained by the method of protocol according to the exemplary embodiment shown in FIG. 1;

FIG. 3 shows a comparison of the results obtained by the method of protocols of the exemplary embodiment shown in FIG. 1 and the related art protocol;

FIG. 4 is a flow chart depicting a method of protocol of measuring an amount of nuclear DNA by using a freeze-dried cell treatment solution according to an exemplary embodiment of present invention; and

FIG. 5 is a flow chart showing another method of protocol of measuring an amount of nuclear DNA by using a cell treatment solution and according to an exemplary embodiment of the present invention.

### DETAILED DESCRIPTION OF THE INVENTION

The present invention will now be described more fully with reference to the accompanying drawings in which exemplary methods of protocol of measuring the amount of nuclear DNA are depicted.

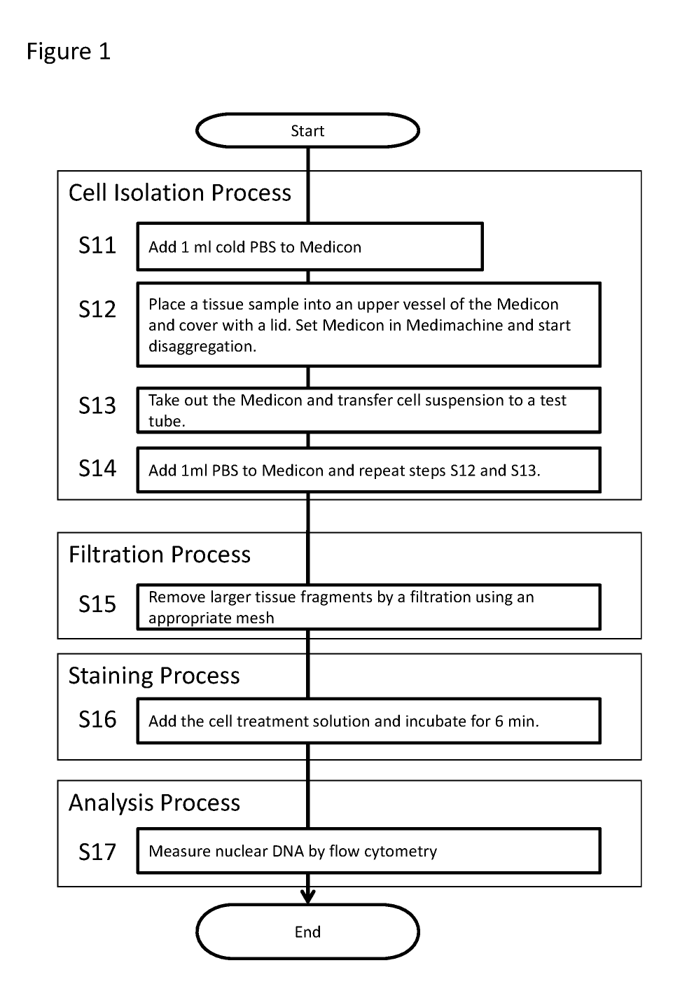

In the exemplary method shown in FIG. 1, a tissue disaggregating system for mechanically separating tissues is employed. For example, devices such as Medimachine and Medicon (manufactured by As One Corporation) can be used for this purpose.

One ml cold phosphate buffered saline (PBS) may be poured into the above-mentioned Medicon (S11), and a tissue segment that may be prepared in advance is put into an upper vessel of the Medicon and covered with a lid.

The Medicon may then be set in the Medimachine and subjected to tissue disaggregation (S12). This tissue disaggregation, for example, may be carried out at a number of rotations of 100 rpm of a rotary knife for 10 seconds, although other number of rotation and time combinations are not precluded by the example.

After the treatment of the step S12, the Medicon may be drawn out, and cell suspension may be transferred to a test tube (S13). One ml of cold PBS may be poured into the Medicon (S14), and then steps S12 and S13 may be repeated. The above treatment is known herein as a cell isolation process.

The cell suspension obtained in the above-mentioned cell isolation process using the mechanical disaggregation may be filtered by a mesh with a mesh diameter of, for example, 100 μm (S15). This treatment is known herein as a filtration process.

Next, the cell treatment solution may be added to the cell suspension obtained from the filtration process. An exemplary composition of the cell treatment solution may comprise, for example, 0.03% Triton X-100, 0.03% RNase and 60 μg/ml propidium iodide in phosphate buffer, however, other surfactants, for example; Tween 20 (polyoxyethylene sorbitan monolaurate) and NP-40 (polyoxyethylene (9) octylphenyl ether) and other fluorescent dyes, for example; DAPI (4′,6-diamidino-2-phenylindole) can also be used.

The mixed cell suspension may be incubated for a prescribed time such as 6 minutes (S16). This treatment is known herein as a staining process and the three processes that are carried out in the three sequential steps in the related art method move forward simultaneously, shortening the time for the reaction to about 6 minutes in the exemplary embodiment.

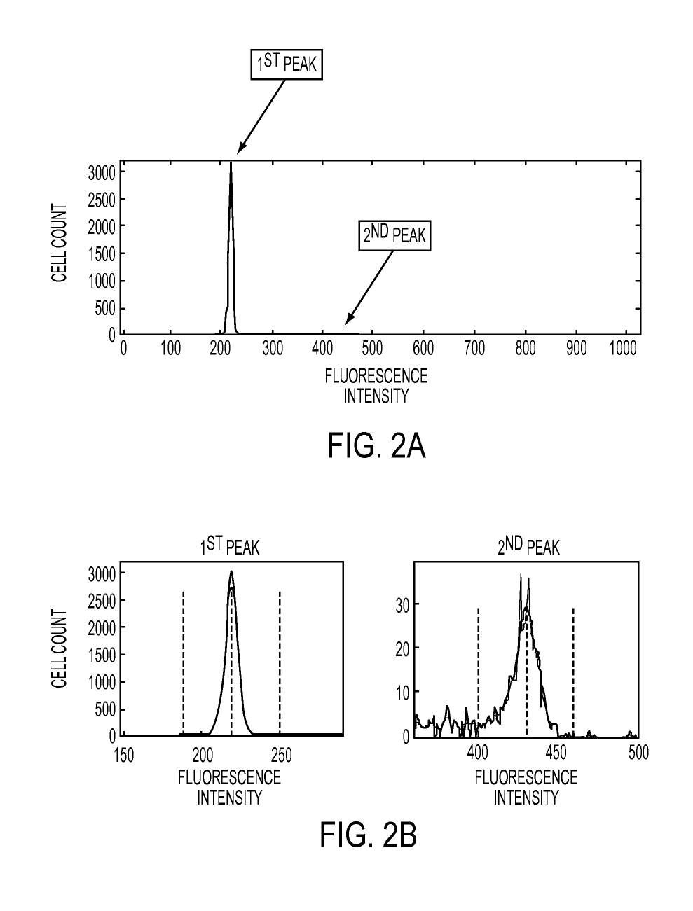

After the above-mentioned staining process, the stained cell suspension may be provided to a flow cytometer for the measurement of the amount of nuclear DNA. (S17). The results may be shown as a histogram, which is a graph of cell count on the y-axis and the fluorescence intensity on the x-axis as shown in FIG. 2 at the top of the Figure. The bottom of FIG. 2 shows an enlargement of the first peak (G0/G1 period) and the second peak (G2/M period).

The quality of the data obtained using the cell treatment solution and the exemplary methods described in the present invention were investigated by comparing the results using a related art method. A set of 6 samples was prepared using the related art method and another set of 6 samples was prepared using the method in the present invention, and all 12 samples were provided to flow cytometry measurement.

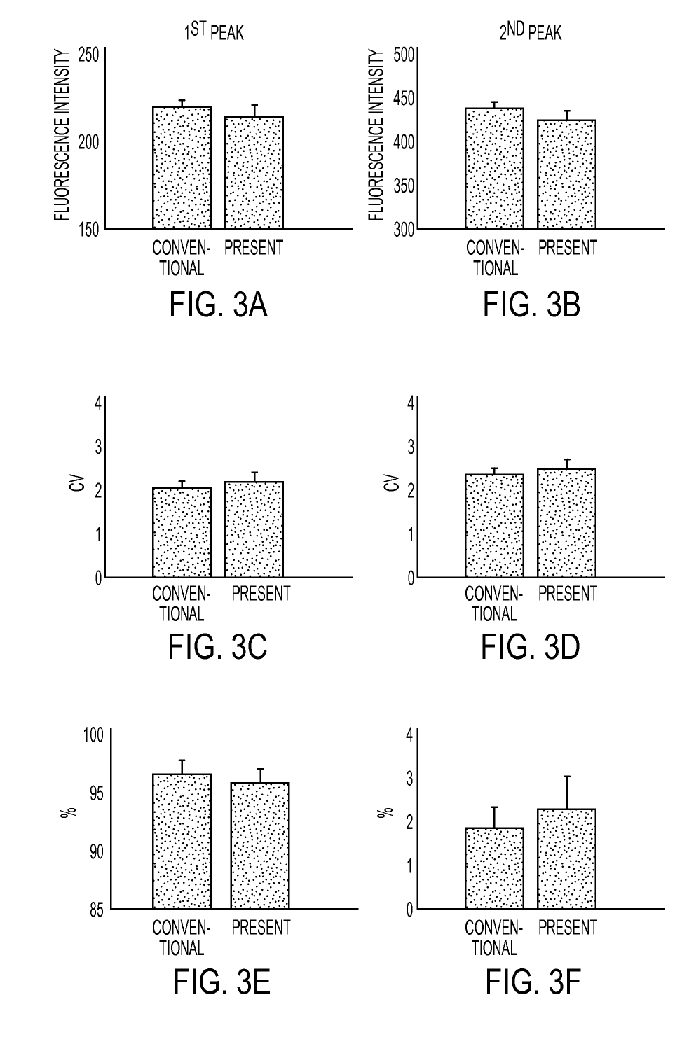

Data from all 12 samples showed a larger first peak and a much smaller second peak as depicted in FIG. 2. For each peak, average fluorescence intensity, CV, and percentage of the cells included in each peak were calculated for each set of 6 samples provided by the two preparations. As seen in FIG. 3, both preparations provided at least comparable results in the average fluorescence intensity, CV, and the percentage of the cells included in each peak.

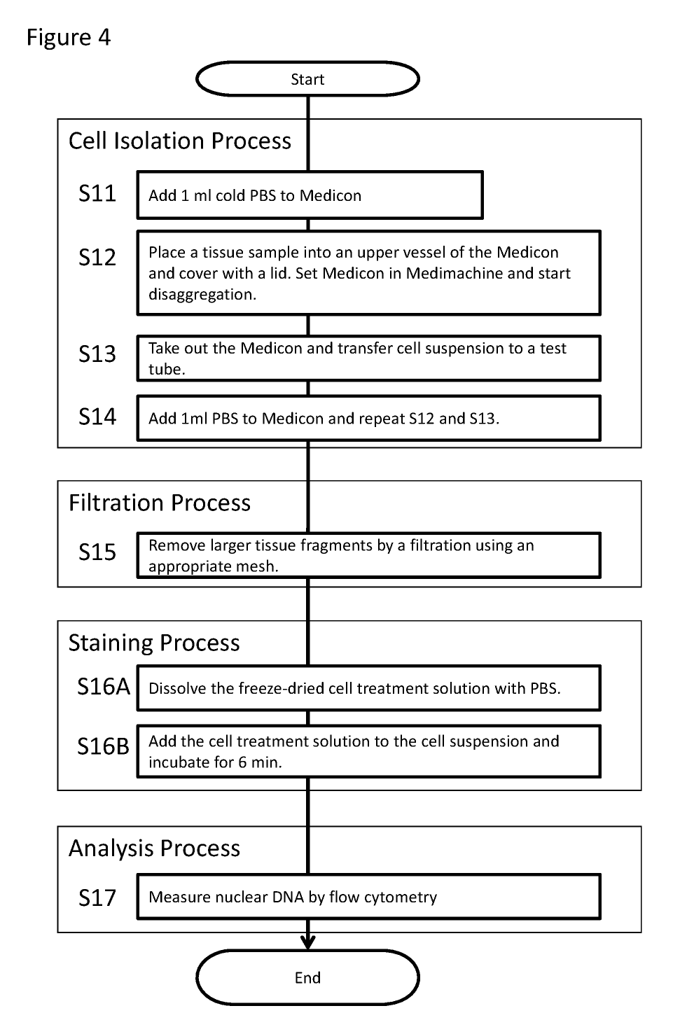

FIG. 4 shows another exemplary method of protocol of the present invention where a freeze-dried cell treatment solution is dissolved with a PBS preparing the cell treatment solution in the step S16A.

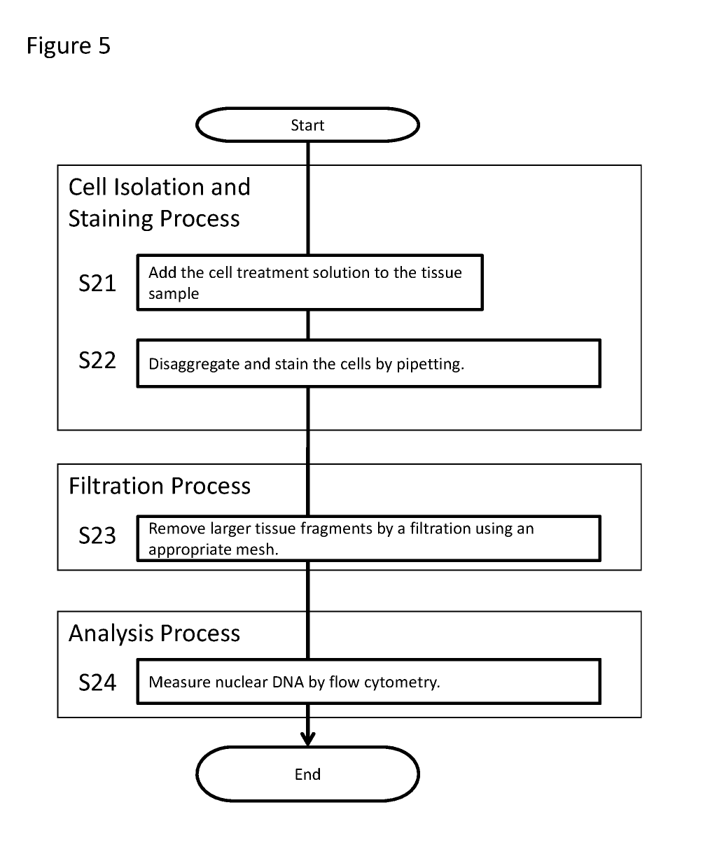

FIG. 5 shows yet another exemplary method of protocol of the present invention where the cell treatment solution is added to the tissue sample in the first step (S21) and disaggregated and stained by pipetting (S22). Next, the mixture from the step S22 is filtered by a mesh to generate stained cell suspension (S23: filtration process). The stained cell suspension obtained in the filtration process is provided to a chromosome analysis using a flow cytometry (S24: analysis process).

If a freeze dried cell treatment solution is used in this method of protocol, it is dissolved with PBS in advance and the tissue sample is added to the cell treatment solution (S22).

While this invention has been particularly shown and described with reference to exemplary embodiments thereof, it will be understood by those skilled in the art that various changes in form and details may be made therein without departing from the spirit and scope of the invention as defined by the appended claims.

What is claimed is:

1. A cell treatment solution for preparing a stained cell suspension that is provided to a measurement of nuclear DNA by flow cytometry, the cell treatment solution comprising:
a surfactant;
RNase; and
fluorescent dye.

2. The cell treatment solution according to claim 1, wherein the surfactant is one of a non-ionic surfactant and a zwitterionic surfactant.

3. The cell treatment according to claim 1, wherein the surfactant is one of an anionic surfactant and a cationic surfactant.

4. The cell treatment solution according to claim 1, wherein the surfactant includes one of Triton X-100, Tween 20 (polyoxyethylene sorbitan monolaurate), and NP-40 (polyoxyethylene (9) octylphenyl ether), or a combination thereof.

5. The cell treatment solution according to claim 4, wherein the Triton X-100 is a 0.03% Triton X-100 concentration.

6. The cell treatment solution according to claim 1, wherein the fluorescent dye includes a propidium iodide in phosphate buffer.

7. The cell treatment solution according to claim 6, wherein the propidium iodide in phosphate buffer is a 60 μg/ml propidium iodide in phosphate buffer concentration.

8. The cell treatment solution of claim 1, where in the solution is freeze-dried.

9. A method of preparing stained cell suspension that is provided to a measurement of nuclear DNA by flow cytometry, the method comprising:
adding a tissue sample to a cell treatment solution comprising a surfactant, RNase, and fluorescent dye;
disaggregating the tissue sample; and
filtering the disaggregated tissue sample.

10. The method of preparing stained cell suspension according to claim 9, further comprising a step of preparing the cell treatment solution by adding a buffer solution to a freeze-dried cell treatment solution comprising the surfactant, the RNase, and the fluorescent dye.

11. The method of preparing stained cell suspension according to claim 10, wherein the steps of adding the buffer solution to the freeze-dried cell treatment solution and disaggregating the tissue sample are performed sequentially by a self-acting mechanism.

12. The method of preparing stained cell suspension according to claim 9, wherein the surfactant is one of a non-ionic surfactant and zwitterionic surfactant.

13. The method of preparing stained cell suspension according to claim 9, wherein the surfactant is one of an anionic surfactant and a cationic surfactant.

14. The method of preparing stained cell suspension according to claim 9, wherein the surfactant includes one of Triton X-100, Tween 20 (polyoxyethylene sorbitan monolaurate), and NP-40 (polyoxyethylene (9) octylphenyl ether) or a combination thereof.

15. The method of preparing stained cell suspension according to claim 14, wherein the Triton X-100 is a 0.03% Triton X-100 concentration.

16. A method of preparing stained cell suspension that is provided to a measurement of nuclear DNA by flow cytometry, the method comprising:
disaggregating tissue sample;
preparing cell suspension by filtering the disaggregated tissue sample; and
adding a cell treatment solution comprising a surfactant, RNase, and fluorescent dye.

17. The method of preparing stained cell suspension according to claim 16, further comprising a step of preparing the cell treatment solution by adding a buffer solution to a freeze-dried cell treatment solution comprising the surfactant, the RNase, and the fluorescent dye.

18. The method of preparing stained cell suspension according to claim 16, wherein the surfactant is one of a non-ionic surfactant and zwitterionic surfactant.

19. The method of preparing stained cell suspension according to claim 16, wherein the surfactant is one of an anionic surfactant and a cationic surfactant.

20. The method of preparing stained cell suspension according to claim 16, wherein the surfactant includes one of Triton X-100, Tween 20 (polyoxyethylene sorbitan monolaurate), and NP-40 (polyoxyethylene (9) octylphenyl ether), or a combination thereof.

21. The method of preparing stained cell suspension according to claim 20, wherein the Triton X-100 is a 0.03% Triton X-100 concentration.
